(19) United States
(12) Patent Application Publication
(10) Pub. No.: US 2025/0325778 A1
(43) Pub. Date: Oct. 23, 2025

(54) Method for Operating a Sleep Assistant During a Charging Process of a Vehicle, Computer-Readable Medium, System, and Vehicle

(71) Applicant: Bayerische Motoren Werke Aktiengesellschaft, Muenchen (DE)

(72) Inventor: Alexander Busch, Planegg (DE)

(21) Appl. No.: 18/855,306

(22) PCT Filed: Feb. 17, 2023
(86) PCT No.: PCT/EP2023/054024

§ 371 Date: Oct. 8, 2024

(30) Foreign Application Priority Data
Apr. 27, 2022 (DE) 10 2022 110 158.4

(51) Int. Cl. A61M 21/00; B60L 53/62; G01C 21/34

(52) U.S. Cl. CPC A61M 21/00; B60L 53/62; G01C 21/3469; A61M 2021/0027; A61M 2021/0044; A61M 2021/0083; A61M 2205/3306; B60L 2240/62; B60L 2240/68; B60L 2250/10; B60L 2250/14; B60L 2250/22

(57) ABSTRACT

A method for operating a sleep assistant during a charging process of a vehicle includes receiving a predicted charging time and determining a preferred sleep duration of a vehicle occupant. The method also includes determining a wake-up time of the vehicle occupant depending on at least one of the group consisting of the predicted charging time and the preferred sleep duration of the vehicle occupant. The method further includes adjusting the wake-up time if an extension of the predicted charging time is detected by the vehicle, and providing a wake-up setting to the vehicle occupant depending on the wake-up time or the adjusted wake-up time.

The present application is the U.S. national phase of PCT Application PCT/EP2023/054024 filed on Feb. 17, 2023, which claims priority of German patent application No. 10 2022 110 158.4 filed on Apr. 27, 2022, the entire contents of which are incorporated herein by reference.

### TECHNICAL FIELD

The disclosure relates to methods and apparatus for operating a sleep assistant during a vehicle charging process, and a vehicle containing a system for operating a sleep assistant during a vehicle charging process.

### BACKGROUND

A charging process at a charging station, such as a fast charging station, takes than usually longer a conventional refueling process. It is often not possible to work at a location of the charging station because the charging station is located outside built-up areas or the charging process at the charging station takes place outside the regular business hours of shops at the location of the charging station. However, the driver wants to make good use of the time during the charging process.

There is a need, therefore, for methods or apparatus that help a driver make use of time during a charging process.

### SUMMARY

This need as well as others by operating a sleep assistant efficiently during a vehicle charging process, so as to efficiently assist a user of the vehicle during a short sleep during the charging process.

A first aspect is characterized by a method for operating a sleep assistant during a vehicle charging process. The method can be computer-implemented method and/or a control device-implemented method. The sleep assistant can be a vehicle function that includes a digital assistant that assists the user while sleeping during charging. For example, the sleep assistant can be configurable. Furthermore, the sleep assistant can monitor the user's falling asleep and/or initiate and/or supervise the user's wake-up process. The method involves receiving a predicted charging time and determining a preferred sleep duration of an occupant of a vehicle. For example, the preferred sleep duration can correspond to the predicted charging time. Preferably, the preferred sleep duration is a duration of a short sleep, for example 10, 20, . . . 50, or 60 minutes. The method also includes determining a wake-up time of the occupant depending on the predicted charging time and/or the preferred sleep duration of the occupant of the vehicle. The method adjusts the wake-up time if an extension of the predicted charging time is detected by the vehicle. Finally, the method provides a wake-up setting to the occupant of the vehicle depending on the wake-up time or the adjusted wake-up time.

Advantageously, the method can efficiently assist a user of the vehicle while sleeping during a charging process. The wake-up time can be adjusted automatically, so that the user has the opportunity to sleep during the entire charging time and not be woken up too early by the vehicle. The wake-up setting can help the user to be woken up in such a way that the user can drive the vehicle awake and safely again. A tired user of the vehicle can thus recover during the charging process and does not have to travel the rest of a distance along a planned navigation route while tired or even overtired. The time during the charging process can be used sensibly by the occupant of the vehicle.

According to an advantageous design, determining the preferred sleep duration of the occupant of the vehicle may include providing a suggestion message to an occupant of the vehicle to activate the sleep assistant during the charging of the vehicle. The suggestion message is preferably provided if an occupant is detected on a seat of the vehicle during the charging process and/or if a position of the vehicle indicates that the vehicle is not near a home position of the vehicle. Furthermore, the suggestion message may preferably be provided in darkness and/or may include one or more suggestions for a sleep duration, in particular a sleep duration of a short sleep. This allows the preferred sleep duration to be determined efficiently and/or easily by an occupant of the vehicle.

According to a further, advantageous design, the determination of the preferred sleep duration of the occupant of the vehicle may also include receiving an input from the occupant of the vehicle in response to the suggestion message to activate the sleep assistant, wherein the occupant's input preferably includes the preferred sleep duration of the occupant of the vehicle.

After receiving the operating input of the occupant of the vehicle, the sleep assistant can be activated. This can be used to activate the sleep assistant efficiently.

According to a further advantageous design, the method can also include recording a time when the occupant of the vehicle falls asleep by an interior camera of the vehicle, and determining the wake-up time of the occupant depending on the predicted charging time, the preferred sleep duration, and/or the time when the occupant of the vehicle falls asleep. This allows the wake-up time to be determined more precisely by the sleep assistant.

According to a further, advantageous design, the method can also include receiving a traffic-related delay during the charging process of the vehicle, wherein the traffic-related delay is preferably a traffic-related delay along a current navigation route of the vehicle, and an adjustment of the wake-up time depending on the traffic-related delay. This allows the wake-up time to be determined more accurately and/or flexibly by the sleep assistant.

According to a further advantageous design, the wake-up setting of the vehicle can be an audiovisual wake-up setting and/or the wake-up setting of the determined wake-up time can be different from the wake-up setting of the adjusted wake-up time. This allows the waking up of the occupant to be efficiently controlled by the sleep assistant.

According to a further, advantageous design, the method can also include recording a wake-up time of the occupant of the vehicle by the interior camera of the vehicle, and providing the wake-up setting to the occupant of the vehicle depending on the recorded wake-up time of the occupant of the vehicle. This allows the wake-up setting to be efficiently controlled by the sleep assistant.

According to a further, advantageous design, the predicted charging time can depend on a planned change of a state of charge of a current navigation route of the vehicle.

According to a further, advantageous design, the method may also include adjusting the current navigation route depending on the wake-up time and a current state of charge of the vehicle, wherein the adjustment of the current navigation route preferably includes adjusting one or more further charging processes during the current navigation route. This allows the further navigation route of the vehicle to be efficiently adjusted by the sleep assistant after the occupant has woken up. The occupant can seamlessly continue the journey along the navigation route after the end of his sleep.

A further aspect is characterized by a computer-readable medium for operating a sleep assistant during a vehicle charging process, wherein the computer-readable medium contains instructions which, when implemented on a computer or a control unit of the vehicle, carry out the method described above.

A further aspect is characterized by a system for operating a sleep assistant during a vehicle charging process, wherein the system is designed to perform the method described above.

According to a further aspect, the invention is characterized by a vehicle containing the system described above for operating a sleep assistant during a vehicle charging process.

Further features result from the claims, the FIGURE and the description of the FIGURE. All the features and combinations of mentioned the features above in description as well as the features and combinations of features mentioned below in the description of the FIGURE and/or shown in the FIGURE alone can be used not only in the respective specified combination, but also in other combinations or on their own.

The above-described features and advantages, as well as others, will become more readily apparent to those of ordinary skill in the art by reference to the following detailed description and accompanying drawings.

### BRIEF DESCRIPTION OF THE DRAWING

FIG. 1 shows an exemplary method for operating a sleep assistant during a vehicle charging process.

### DETAILED DESCRIPTION

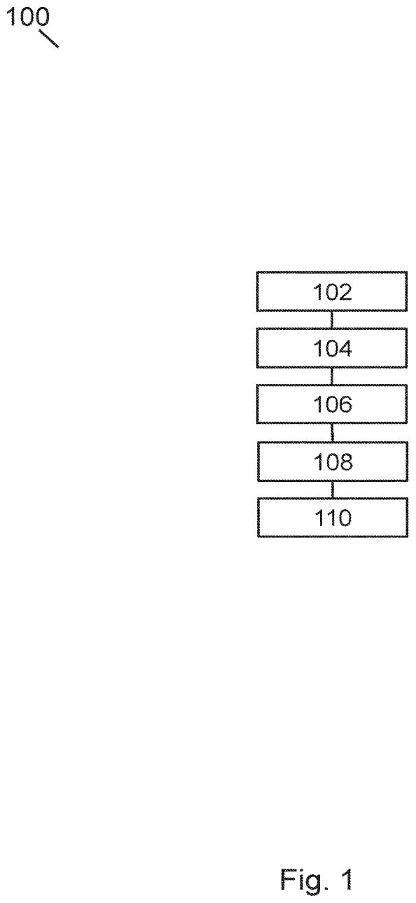

FIG. 1 shows an exemplary method 100 for operating a sleep assistant during a vehicle charging process. The sleep assistant can supervise a sleep process of an occupant of the vehicle, for example a driver of the vehicle, during the charging process of the vehicle and/or can automatically wake up the occupant.

The method 100 can receive 102 a predicted charging time. The predicted charging time may depend on a change of charge that can be achieved during the charging process. The change of charge can be configurable by the occupant of the vehicle and/or predetermined by a navigation route for battery-electric vehicles. For example, the change of charge can include a change in a state of charge from an initial battery state of charge, such as 10%, to a target battery state of charge, such as 80%. In addition or alternatively, the occupant of the vehicle can configure a charging time individually. For example, the occupant of the vehicle can specify that the vehicle will be charged for a predetermined charging period, such as 10, 20, 30, 40, etc., 60 minutes.

Furthermore, the method 100 can determine 104 a preferred sleep duration of the occupant of the vehicle. The preferred sleep duration is preferably a duration of a short sleep. The duration of a short sleep can preferably be set between 10 and 45 minutes, but no more than 60 minutes, by the occupant of the vehicle. For example, the method can determine preferred sleep duration depending on the predicted charging time and/or an individually configured charging time of the occupant. In addition or alternatively, the occupant of the vehicle can configure a preferred sleep duration. To this end, the procedure may suggest one or more sleep durations to the occupant of the vehicle. The preferred sleep duration can be determined when an in-vehicle information system is switched off. For example, the preferred sleep duration can be determined in a farewell display when the internal information system of the vehicle is shut down.

In addition, the method 100 can supervise the occupant when falling asleep after determining the preferred sleep duration. For this purpose, the method 100 can perform a predetermined falling asleep setting. The predetermined falling asleep setting can be an audiovisual falling asleep setting. The audio-visual falling asleep setting may include signals to one or more lighting components in an interior of the vehicle that control the color and/or brightness of the lighting components so that falling asleep of the occupant is assisted. Furthermore, the audiovisual falling asleep setting can play predetermined music and/or a predetermined sequence of sounds that supports the occupant of the vehicle as they fall asleep. A camera in the interior of the vehicle can be used to determine the time when the occupant of the vehicle falls asleep. If a time of falling asleep is determined by the camera, the predetermined falling asleep setting of the vehicle can be ended.

The method 100 can determine 106 a wake-up time of the occupant depending on the predicted charging time and/or the preferred sleep duration of the occupant of the vehicle. The wake-up time can also be determined depending on the time of falling asleep.

Furthermore, the method 100 can adjust 108 the wake-up time if an extension of the predicted charging time is detected by the vehicle. Preferably, the method can adjust the wake-up time while the occupant of the vehicle is sleeping. The wake-up time can also be adjusted to wait for a traffic incident on a navigation route to subside. If the wake-up time has been adjusted due to a traffic incident, the method 100 can also extend the charging time at least to the target state of charge.

After the occupant wakes up, a journey can be continued with the target state of charge and without any traffic disruption.

Finally, the method 100 can provide a wake-up setting to the occupant of the vehicle depending on the wake-up time or the adjusted wake-up time. If the wake-up time has been adjusted by the method, the method 100 can provide the wake-up to the occupant of the vehicle depending on the adjusted wake-up time. If the wake-up time has not been adjusted by the method, the method 100 can provide the wake-up setting to the occupant of the vehicle depending on the wake-up time. The wake-up setting can wake up the occupant of the vehicle gently. Similarly to the falling asleep setting, the wake-up setting can include signals to one or more light components in an interior of the vehicle that control a color and/or brightness of the lighting components and/or play predetermined music and/or a predetermined sequence of sounds. In addition, the wake-up setting can provide a message to the occupant of the vehicle informing the occupant of an adjustment of the wake-up time and/or one or more reasons for doing so.

Advantageously, the method can efficiently assist an occupant of the vehicle, in particular the driver of the vehicle, while sleeping during a charging process. The driver of the vehicle can continue the journey awake and refreshed by sleep. Driver fatigue can thus be efficiently reduced and road safety can be efficiently increased, especially during long journeys and/or journeys at night. By adjusting the wake-up time, the sleep duration of the occupant of the vehicle can be controlled more efficiently. The occupant can thus sleep longer, with the target state of charge, and/or continue the journey without a traffic delay.

### LIST OF REFERENCE SIGNS

100 Method
102 Receiving a charging time
104 Determining a preferred sleep duration
106 Determining a wake-up time
108 Adjusting the wake-up time
110 Providing a wake-up setting

1.-12. (canceled)

13. A method for operating a sleep assistant during a charging process of a vehicle, the method comprising:
receiving a predicted charging time;
determining a preferred sleep duration of a vehicle occupant;
determining a wake-up time of the vehicle occupant depending on at least one of the group consisting of the predicted charging time and the preferred sleep duration of the vehicle occupant;
adjusting the wake-up time if an extension of the predicted charging time is detected by the vehicle; and
providing a wake-up setting to the vehicle occupant depending on the wake-up time or the adjusted wake-up time.

14. The method as claimed in claim 13, wherein determining the preferred sleep duration of the vehicle occupant includes:
providing a suggestion message to the vehicle occupant to activate the sleep assistant during the vehicle charging process.

15. The method as claimed in claim 14, wherein the suggestion message is provided responsive to a detection of an occupant on a seat of the vehicle during the charging process.

16. The method as claimed in claim 14, wherein the suggestion message is provided if a position of the vehicle indicates that the vehicle is not close to a home position of the vehicle.

17. The method as claimed in claim 14, wherein the suggestion message is provided responsive to a determination of darkness.

18. The method as claimed in claim 14, wherein the suggestion message includes one or more suggestions for a sleep duration.

19. The method as claimed in claim 14, wherein determining the preferred sleep duration of the vehicle occupant further includes:
receiving an operating input from the vehicle occupant in response to the suggestion message to activate the sleep assistant,
wherein the operating input of the occupant preferably includes the preferred sleep duration of the vehicle occupant;
and activating the sleep assistant after receiving the operating input of the vehicle occupant.

20. The method as claimed in claim 19, further comprising:
recording a time when the vehicle occupant falls asleep by an interior camera of the vehicle; and
wherein determining the wake-up time of the vehicle occupant further includes determining the wake-up time of the vehicle occupant depending on the time the vehicle occupant falls asleep.

21. The method as claimed in claim 13, further comprising:
recording a time when the vehicle occupant falls asleep by an interior camera of the vehicle; and
wherein determining the wake-up time of the vehicle occupant further includes determining the wake-up time of the vehicle occupant depending on the time the vehicle occupant falls asleep.

22. The method as claimed in claim 13, further comprising:
receiving traffic-related delay information during the charging process of the vehicle; and
adjusting the wake-up time depending on the traffic-related delay information.

23. The method as claimed in claim 13, wherein the wake-up setting of the vehicle is an audiovisual wake-up setting.

24. The method as claimed in claim 23, wherein the wake-up setting of the determined wake-up time is different from the wake-up setting of the adjusted wake-up time.

25. The method as claimed in claim 13, wherein the wake-up setting of the determined wake-up time is different from the wake-up setting of the adjusted wake-up time.

26. The method as claimed in claim 13, further comprising:
recording of a wake-up time of the vehicle occupant by the interior camera of the vehicle; and
providing the wake-up setting to the vehicle occupant depending on the recorded wake-up time of the vehicle occupant.

27. The method as claimed in claim 13, wherein the predicted charging time is dependent on a planned change of a state of charge of a current navigation route of the vehicle.

28. The method as claimed in claim 27, further comprising:
adjusting the current navigation route depending on the wake-up time and a current state of charge of the vehicle, wherein adjusting the current navigation route preferably includes adjusting one or more further charging processes during the current navigation route.

29. A non-transitory computer-readable medium for operating a sleep assistant during a vehicle charging process, wherein the computer-readable medium contains instructions which, when implemented on a computer or a control unit of the vehicle, carry out the method as claimed in claim 13.

30. A system for operating a sleep assistant during a charging process of a vehicle, wherein the system is designed to carry out the method as claimed in claim 13.

31. A vehicle containing the system for operating a sleep assistant during a charging process of a vehicle as claimed in claim 30.
